(19) United States
(12) Patent Application Publication
(10) Pub. No.: US 2025/0352256 A1
(43) Pub. Date: Nov. 20, 2025

(54) MAPPING AND ABLATING CATHETERS USING FLEXIBLE CIRCUIT BOARDS ON SUPPORT MEMBERS

(71) Applicant: Boston Scientific Scimed, Inc., Maple Grove, MN (US)

(72) Inventors: Nathan Paul HAGSTROM, Blaine, MN (US); Andrew L. DEKOCK, Ham Lake, MN (US); Cory P. WRIGHT, Woodbury, MN (US); Michael Sean COE, Plymouth, MN (US); Nathaniel ADAMS, New Richmond, WI (US)

(21) Appl. No.: 19/183,643

(22) Filed: Apr. 18, 2025

Related U.S. Application Data
(60) Provisional application No. 63/648,044, filed on May 15, 2024.

(51) Int. Cl. A61B 18/00

(52) U.S. Cl. CPC A61B 18/00; A61B 2018/00077; A61B 2018/00083; A61B 2018/0016; A61B 2018/00178; A61B 2018/00214; A61B 2018/00351; A61B 2018/00577; A61B 2018/00613

(57) ABSTRACT

A catheter for ablating cardiac tissue through irreversible electroporation, the catheter comprising a flexible circuit having a plurality of flex circuit branches and including an outwardly-facing ablation electrode including a plurality of ablation electrode branches extending proximally along a portion of a respective one of the flex circuit branches, and a support member having a plurality of support member branches, wherein the support member includes an electrically conductive base member covered by an electrically insulative coating, wherein each of the flex circuit branches is secured to a respective one of the support member branches, and the ablation electrode is electrically coupled to the electrically conductive base member.

### CROSS REFERENCE TO RELATED APPLICATIONS

The present application claims priority to U.S. Provisional Patent Application Ser. No. 63/648,044, filed May 15, 2024, the entire disclosure of which is hereby incorporated by reference in its entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. There is a continuing need for improved devices and methods for performing cardiac tissue ablation through irreversible electroporation.

### SUMMARY

In Example 1, a catheter for use in ablating cardiac tissue through irreversible electroporation, the catheter comprising a tubular outer shaft having a proximal end and an opposite distal end, and an electrode assembly extending distally from the distal end of the outer shaft. The electrode assembly defines a distally located central hub portion and a plurality of splines each including a distal end portion extending proximally from the central hub portion, a proximal end portion attached to and constrained by the outer shaft, and an intermediate portion between the proximal end portion and the distal end portion. The electrode assembly comprises a flexible circuit and a support member. The flexible circuit has a flex circuit hub and a plurality of flex circuit branches integrally formed with and extending proximally from the flex circuit hub, the flexible circuit further including an outwardly-facing ablation electrode including a plurality of ablation electrode branches extending proximally along a portion of a respective one of the flex circuit branches and terminating in an ablation electrode proximal end. The support member has a support member hub and a plurality of support member branches extending proximally from the support member hub, wherein the support member includes an electrically conductive base member covered by an electrically insulative coating, wherein each of the flex circuit branches is secured to a respective one of the support member branches.

In Example 2, the catheter of Example 1, wherein the flexible circuit includes a flex circuit hub and the plurality of flex circuit branches are integrally formed with and extend proximally from the flex circuit hub.

In Example 3, the catheter of Example 2, wherein the ablation electrode includes an ablation electrode hub portion located on the flex circuit hub, and the plurality of ablation electrode branches are integrally formed with the ablation electrode hub portion.

In Example 4, the catheter of any of Examples 1-3, wherein the electrically insulative coating comprises a silicone, parylene, or polyvinylidene fluoride coating.

In Example 5, the catheter of any of Examples 1-4, wherein the electrically insulative coating is deposited via a process including spray coat, dip coat, chemical vapor deposition, and atomic layer deposition.

In Example 6, the catheter of any of Examples 1-3, wherein the electrically insulative coating has a laminated structure including an upper dielectric layer, a lower dielectric layer, and an adhesive layer.

In Example 7, the catheter of Example 6, wherein the adhesive layer includes an upper adhesive layer portion disposed over an upper surface of the conductive base member, and a lower adhesive layer portion disposed over a lower surface of the conductive base member, and wherein the upper dielectric layer is disposed over the upper adhesive layer portion, and the lower dielectric layer is disposed over the lower adhesive layer portion.

In Example 8, the catheter of Example 7, wherein the upper and lower dielectric layers each comprise a polyimide film or a liquid crystal polymer film.

In Example 9, the catheter of Example 7, wherein the upper and lower adhesive layer portions each comprise an acrylic adhesive film.

In Example 10, the catheter of either of Examples 8 or 9, wherein the upper and lower dielectric layers and the adhesive layer form dielectric coating extensions extending laterally from opposite lateral edges of the conductive base member.

In Example 11, the catheter of any of Examples 1-3, wherein the electrically insulative coating is overmolded to the electrically conductive base member.

In Example 12, the catheter of Example 11, wherein the electrically insulative coating is formed of a moldable elastomeric material.

In Example 13, the catheter of Example 11, wherein the moldable elastomeric material is a polyether block amide or silicone.

In Example 14, the catheter of any of Examples 1-3, wherein the plurality of flex circuit branches comprises a liquid crystal polymer material.

In Example 15, the catheter of any of Examples 1-3, wherein the electrically insulative coating comprises a liquid crystal polymer material and the electrically insulative coating is an upper layer that is mechanically bonded to a lower layer using a process of reflowing the liquid crystal polymer material.

In Example 16, the catheter of Example 15, wherein the upper and lower dielectric layer are in direct contact with the conductive base member.

In Example 17, the catheter of Example 16, wherein the upper dielectric layer is in direct contact with the plurality of flex circuit branches.

In Example 18, the catheter of any of Examples 1-17, further comprising a plurality of spline sensing electrodes located on each spline.

In Example 19, the catheter of any of Examples 1-18, further comprising a hub sensing electrode centrally located on the central hub portion of the electrode assembly.

In Example 20, a catheter for use in ablating cardiac tissue through irreversible electroporation, the catheter comprising a tubular outer shaft having a proximal end and an opposite distal end, and an electrode assembly extending distally from the distal end of the outer shaft. The electrode assembly defines a distally located central hub portion and a plurality of splines each including a distal end portion extending proximally from the central hub portion, and a proximal end portion attached to and constrained by the outer shaft. The electrode assembly comprises a flexible circuit and a support member. The flexible circuit has a flex circuit hub and a plurality of flex circuit branches integrally formed with and extending proximally from the flex circuit hub, the flexible circuit further including an outwardly-facing ablation electrode including a plurality of ablation electrode branches extending proximally along a portion of a respective one of the flex circuit branches and terminating in an ablation electrode proximal end. The support member has a support member hub and a plurality of support member branches extending proximally from the support member hub, wherein the support member includes an electrically conductive base member covered by an electrically insulative coating, wherein each of the flex circuit branches is secured to a respective one of the support member branches, and the ablation electrode is electrically coupled to the electrically conductive base member.

In Example 21, the catheter of Example 20, wherein the electrically insulative coating comprises a silicone, parylene, or polyvinylidene fluoride coating.

In Example 22, the catheter of Example 21, wherein the electrically insulative coating is deposited via a process including spray coat, dip coat, chemical vapor deposition, and atomic layer deposition.

In Example 23, the catheter of Example 20, wherein the electrically insulative coating has a laminated structure including an upper dielectric layer, a lower dielectric layer, and an adhesive layer.

In Example 24, the catheter of Example 23, wherein the adhesive layer includes an upper adhesive layer portion disposed over an upper surface of the conductive base member, and a lower adhesive layer portion disposed over a lower surface of the conductive base member, wherein the upper dielectric layer is disposed over the upper adhesive layer portion, and the lower dielectric layer is disposed over the lower adhesive layer portion.

In Example 25, the catheter of Example 24, wherein the upper and lower dielectric layers each comprise a polyimide film or a liquid crystal polymer film.

In Example 26, the catheter of Example 25, wherein the upper and lower adhesive layer portions each comprise an acrylic adhesive film.

In Example 27, the catheter of Example 26, wherein the upper and lower dielectric layers and the adhesive layer form dielectric coating extensions extending laterally from opposite lateral edges of the conductive base member.

In Example 28, the catheter of Example 20, wherein the electrically insulative coating is overmolded to the electrically conductive base member.

In Example 29, the catheter of Example 28, wherein the electrically insulative coating is formed of a moldable elastomeric material.

In Example 30, the catheter of Example 29, wherein the moldable elastomeric material is a polyether block amide or silicone.

In Example 31, the catheter of Example 20, wherein the plurality of flex circuit branches comprises a liquid crystal polymer material.

In Example 32, the catheter of Example 20, wherein the electrically insulative coating comprises a liquid crystal polymer material and the electrically insulative coating is an upper layer that is mechanically bonded to a lower layer using a process of reflowing the liquid crystal polymer material.

In Example 33, the catheter of Example 30, wherein the upper and lower dielectric layer are in direct contact with the conductive base member, and the upper dielectric layer is in direct contact with the plurality of flex circuit branches.

In Example 34, a catheter for use in ablating cardiac tissue through irreversible electroporation, the catheter comprising a tubular shaft, and a splined electrode assembly comprising a support member comprising an electrically conductive base member covered by an electrically insulative coating, and a flexible circuit secured to the support member, the flexible circuit having a dielectric substrate layer and an ablation electrode disposed on the dielectric substrate layer, wherein the ablation electrode is electrically coupled to the conductive base member.

In Example 35, the catheter of Example 34, wherein the electrically insulative coating includes an upper adhesive layer portion disposed over an upper surface of the conductive base member, a lower adhesive layer portion disposed over a lower surface of the conductive base member, an upper dielectric layer disposed over the upper adhesive layer portion, and a lower dielectric layer disposed over the lower adhesive layer portion.

In Example 36, the catheter of Example 35, wherein the upper and lower dielectric layers each comprise a polyimide film or a liquid crystal polymer film, and the upper and lower adhesive layer portions each comprise an acrylic adhesive film.

In Example 37, the catheter of Example 36, wherein the electrically insulative coating is overmolded to the electrically conductive base member.

In Example 38, the catheter of Example 37, wherein the electrically insulative coating comprises a polyether block amide or silicone coating.

In Example 39, the catheter of Example 34, wherein the plurality of flex circuit branches comprises a liquid crystal polymer material.

In Example 40, the catheter of Example 34, wherein the electrically insulative coating comprises a liquid crystal polymer material and the electrically insulative coating is an upper layer that is mechanically bonded to a lower layer using a process of reflowing the liquid crystal polymer material.

In Example 41, the catheter of Example 40, wherein the upper and lower dielectric layer are in direct contact with the conductive base member, and the upper dielectric layer is in direct contact with the plurality of flex circuit branches.

In Example 42, a catheter for use in ablating cardiac tissue through irreversible electroporation, the catheter comprising a tubular shaft, and an electrode assembly extending from the tubular shaft, the electrode assembly comprising a plurality of splines extending proximally from a distal hub portion, the distal hub portion and the plurality of splines comprising a support member comprising an electrically conductive base member, and an electrically insulating coating disposed over the electrically conductive base member, and a flexible circuit comprising a dielectric substrate secured to the electrically insulative coating, and an ablation electrode disposed on the dielectric substrate layer, wherein the ablation electrode is electrically coupled to the conductive base member.

In Example 43, the catheter of Example 42, wherein the electrically insulative coating has a laminated structure including an upper adhesive layer disposed on an upper surface of the conductive base member, a lower adhesive layer disposed on a lower surface of the conductive base member, an upper dielectric layer disposed on the upper adhesive layer, and a lower dielectric layer disposed on the lower adhesive layer.

In Example 44, the catheter of Example 43, further comprising dielectric coating extensions extending laterally from opposite lateral edges of the conductive base member, each of the dielectric coating extensions comprising an extension of the upper and lower dielectric layers with the upper and lower adhesive layers disposed therebetween.

In Example 45, the catheter of Example 43, wherein the upper and lower dielectric layers each comprise a polyimide film or a liquid crystal polymer film, and the upper and lower adhesive layers each comprise an acrylic adhesive film. While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

In Example 46, the catheter of Example 43, wherein the electrically insulative coating comprises a liquid crystal polymer material, and the electrically insulative coating is an upper layer that is mechanically bonded to a lower layer using a process of reflowing the liquid crystal polymer material, and the upper and lower dielectric layer are in direct contact with the conductive base member.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2A is a perspective illustration of a distal portion of a splined catheter for use in the electrophysiology system of FIG. 1, in accordance with embodiments of the subject matter of the disclosure.

FIGS. 2B-2C are partial plan views an electrode assembly of the splined catheter shown in two-dimensions, in accordance with embodiments of the subject matter of the disclosure.

FIG. 2D is an enlarged plan view of a portion of a spline of the electrode assembly shown in FIG. 2B, in accordance with embodiments of the subject matter of the disclosure.

FIG. 3 is a schematic cross-sectional view of a configuration of a spline of the electrode assembly of FIG. 2B, in accordance with embodiments of the subject matter of the disclosure.

FIG. 4 is a schematic cross-sectional view of another configuration of a spline of the electrode assembly of FIG. 2B, in accordance with embodiments of the subject matter of the disclosure.

FIG. 5A is a schematic cross-sectional view of still another configuration of a spline of the electrode assembly of FIG. 2B, in accordance with embodiments of the subject matter of the disclosure.

FIG. 5B is an isometric illustration of a support member including the spline of FIG. 5A, in accordance with embodiments of the subject matter of the disclosure.

FIG. 6 is a schematic cross-sectional view of still another configuration of a spline of the electrode assembly of FIG. 2B, in accordance with embodiments of the subject matter of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

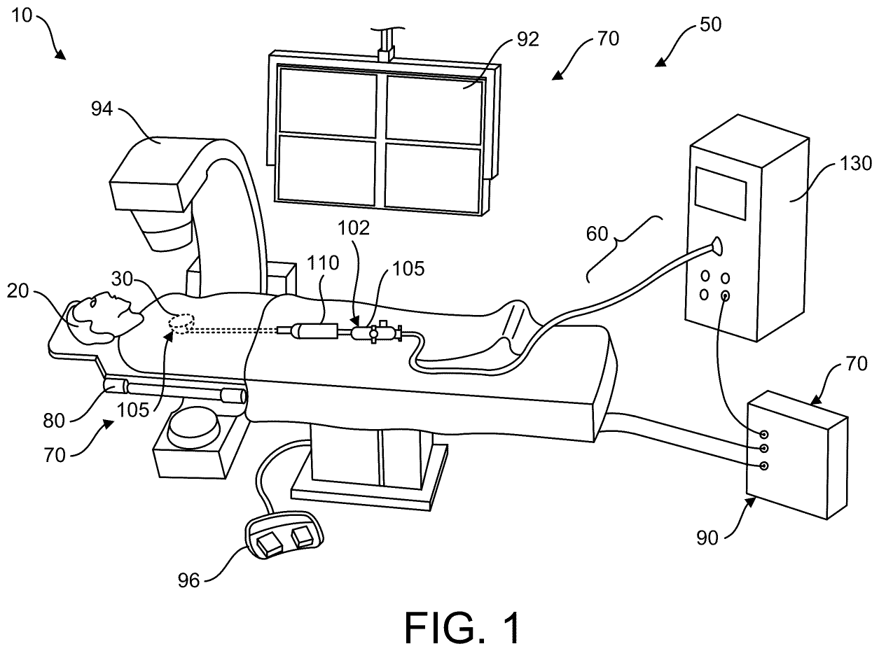

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation catheter system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The electroporation catheter system 60 includes an electroporation catheter 100 having a proximal portion 102 and a distal portion 105, an introducer sheath 110, and an electroporation console 130. Additionally, the electroporation catheter system 60 includes various connecting elements, e.g., cables, umbilicals, and the like, that operate to functionally connect the components of the electroporation catheter system 60 to one another and to the components of the EAM system 70. This arrangement of connecting elements is not of critical importance to the present disclosure, and the skilled artisan will recognize that the various components described herein can be interconnected in a variety of ways.

In embodiments, the introducer sheath 110 is operable to provide a delivery conduit through which the electroporation catheter 100, in particular all or part of the distal portion 105 thereof, can be deployed to the specific target sites within the patient's heart 30.

In embodiments, the electroporation catheter system 60 is configured to deliver electric field energy to targeted tissue in the patient's heart 30 to create tissue apoptosis, rendering the tissue incapable of conducting electrical signals.

The electroporation console 130 is configured to control functional aspects of the electroporation catheter system 60. In embodiments, the electroporation console 130 includes one or more controllers, microprocessors, and/or computers that execute code out of memory to control and/or perform the functional aspects of the electroporation catheter system 60. In embodiments, the memory can be part of the one or more controllers, microprocessors, and/or computers, and/or part of memory capacity accessible through a network, such as the world wide web. In embodiments, the electroporation console 130 includes pulse generator hardware, software and/or firmware configure to generate electrical pulses in predefined waveforms, which are transmitted to electrodes on the electroporation catheter 100 to generate electric fields sufficient to achieve the desired clinical effect, in particular ablation of target tissue through irreversible electroporation. In embodiments, the electroporation console 130 can deliver the pulsed waveforms to the electroporation catheter 100 in a monopolar or bipolar mode of operation, as will be described in further detail herein.

The EAM system 70 is operable to track the location of the various functional components of the electroporation catheter system 60, and to generate high-fidelity three-dimensional anatomical and electro-anatomical maps of the cardiac chambers of interest. In embodiments, the EAM system 70 can be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

Embodiments of the present disclosure provide systems, devices, and methods for selective and rapid application of pulsed electric fields to ablate tissue by irreversible electroporation. Generally, the systems, devices, and methods described herein may be used to generate large electric field magnitudes at desired regions of interest and reduce peak electric field values elsewhere in order to reduce unnecessary tissue damage and electrical arcing. An irreversible electroporation system as described herein may include a signal generator and a processor configured to apply one or more voltage pulse waveforms to a selected set of electrodes of an ablation device to deliver energy to a region of interest (e.g., ablation energy for a set of tissue in a pulmonary vein ostium or antrum). The pulse waveforms disclosed herein may aid in therapeutic treatment of a variety of cardiac arrhythmias (e.g., atrial fibrillation). In order to deliver the pulse waveforms generated by the signal generator, one or more electrodes of the ablation device may have an insulated electrical lead configured for sustaining a voltage potential in the order of several hundred volts to several thousand volts. The electrodes may be independently addressable such that each electrode may be controlled (e.g., deliver energy) independently of any other electrode of the device. In this manner, the electrodes may deliver different energy waveforms with different timing synergistically for electroporation of tissue.

Pulse waveforms for electroporation energy delivery as disclosed herein may enhance the safety, efficiency and effectiveness of energy delivery to tissue by reducing the electric field threshold associated with irreversible electroporation, thus yielding more effective ablative lesions with a reduction in total energy delivered. In some embodiments, the voltage pulse waveforms disclosed herein may be hierarchical and have a nested structure. For example, the pulse waveform may include hierarchical groupings of pulses having associated timescales. In some embodiments, the methods, systems, and devices disclosed herein may comprise one or more of the methods, systems, and devices described in International Application Serial No. PCT/US2016/057664, filed on Oct. 19, 2016, and titled “SYSTEMS, APPARATUSES AND METHODS FOR DELIVERY OF ABLATIVE ENERGY TO TISSUE,” the contents of which are hereby incorporated by reference in its entirety.

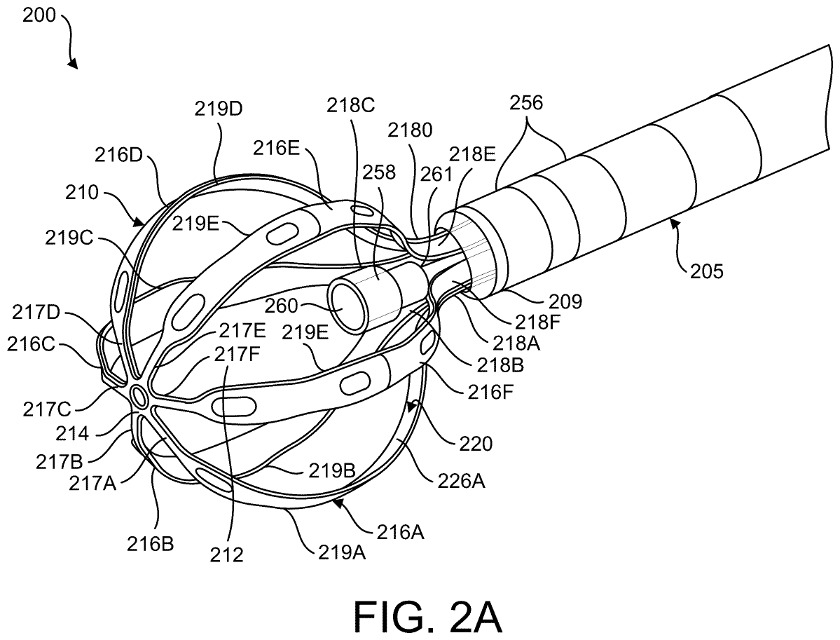

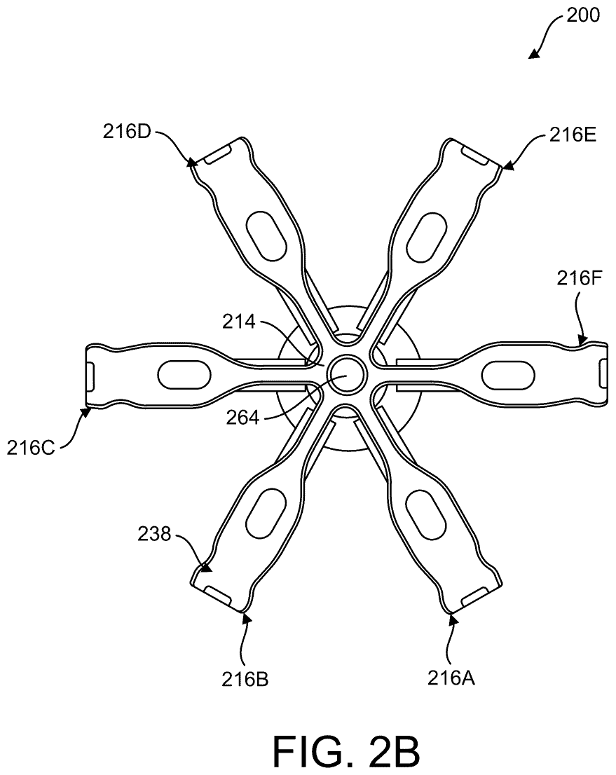

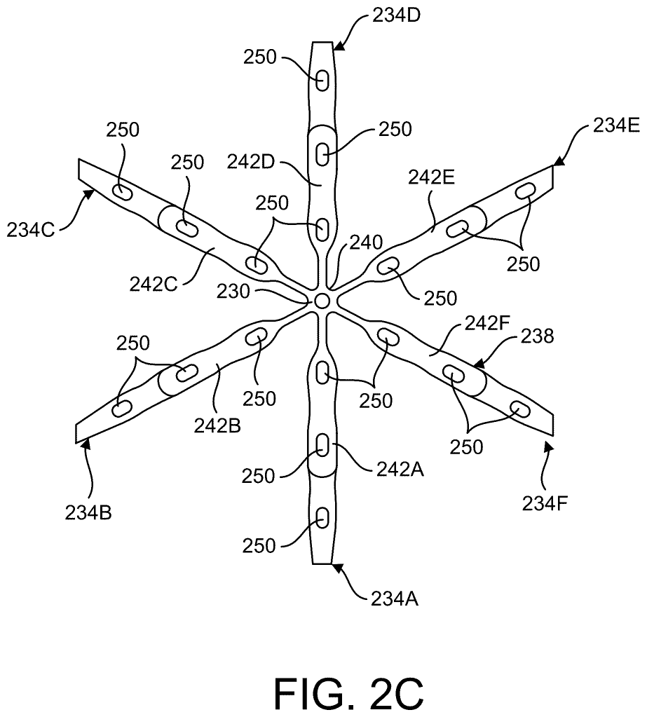

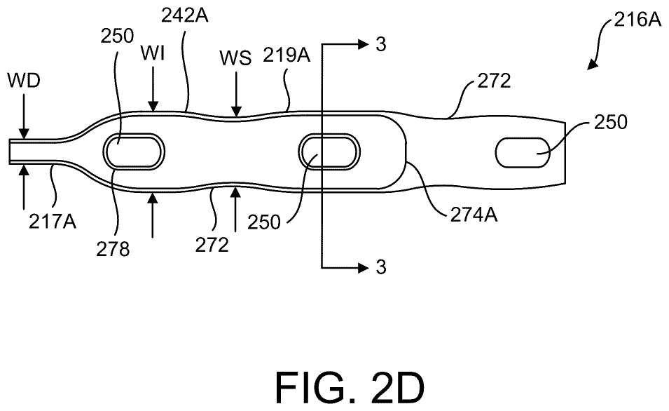

FIGS. 2A and 2B are partial perspective and end view illustrations, respectively, of an electroporation catheter 200 having a catheter distal portion 205 according to an embodiment of the present disclosure. The electroporation catheter 200 corresponds to the electroporation catheter 100 described with respect to FIG. 1. The electroporation catheter 200 has a tubular outer shaft 202 having a shaft distal end 209, and an electrode assembly 210 extending distally from the distal end 209 of the outer shaft 202. In embodiments, the electrode assembly 210 is configured to self-expand from a collapsed configuration when constrained within a delivery sheath to a pre-defined expanded configuration defining an inner space 212. As will be explained in greater detail herein, the electrode assembly 210 comprises an ablation electrode configured to receive pulsed electrical signals/waveforms from the electroporation console 130 (FIG. 1), thereby creating pulsed electric fields sufficient for ablating target tissue via irreversible electroporation. Additionally, the electrode assembly 210 further includes a plurality of mapping and sensing electrodes configured for, among other things, sensing cardiac electrical signals, localization of the electrode assembly 210 within the patient anatomy (e.g., via the EAM system 70 of FIG. 1), and determining proximity to target tissue within the anatomy.

Overall, the electrode assembly 210 and other electrode assembly embodiments described herein within the scope of the present disclosure, is primarily designed for the creation of relatively localized ablation lesions (i.e., focal lesions), as compared to relatively large diameter circumferential lesions created in pulmonary vein isolation procedures. However, the skilled artisan will appreciate that the teachings of the present disclosure can be readily adapted for a catheter capable of large diameter circumferential lesions. The designs of the various electrode assembly embodiments described herein can provide the clinician with a wide range of capabilities for monopolar and bipolar focal pulsed field ablation of cardiac tissue, combined with the ability to perform localized (i.e., at the location of the delivery of pulsed field ablative energy), high fidelity sensing of cardiac tissue, e.g., for lesion or conduction block assessment, tissue contact determinations, and the like.

FIG. 2C is a partial plan view of the electrode assembly 210 of the electroporation catheter 200 shown, shown in two-dimensions to illustrate the layout of the electrode assembly 210. Referring to FIGS. 2A-2C together, in the illustrated embodiment, the electrode assembly 210 as a whole has a distally-located central hub portion 214 and a plurality of splines 216A-216F extending proximally from the central hub portion 214. As further shown, each respective spline 216A-216F has a distal end portion 217A-217F, a proximal end portion 218A-218F, and an intermediate portion 219A-219F extending between the distal end portion 217A-217F and the proximal end portion 218A-218F. As shown, each of the proximal end portions 218A-218F is attached to and constrained by the distal end 209 of the outer shaft 202. As further shown, in the illustrated embodiment, the intermediate portion 219A-219F of each spline 216A-216F has a lateral width that is greater than the lateral width of each of the respective distal end portions 217A-217F. In embodiments, the particular geometry of the splines 216A-216F and the related components, e.g., ablation and mapping electrodes, is optimized to provide desired mechanical and therapeutic/diagnostic capabilities.

In the illustrated embodiment, the splines 216A-216F are composed of a support member 220 and a flexible circuit 222 secured to and disposed over an outer surface of the support member 220. The support member 220 functions, among other things, as a primary structural support of the electrode assembly 210, and thus primarily defines the mechanical characteristics of the electrode assembly 210. In embodiments, the support member 220 is formed from a superelastic material (metal or polymer) to provide desired mechanical/structural properties to the electrode assembly 210. In embodiments, the support member 220 is formed from a superelastic metal alloy, e.g., a nickel-titanium alloy.

The support member 220 includes a support member hub 224 and a plurality of support member branches (for ease of illustration, only support member branch 226A is labeled in FIG. 2A). In embodiments, the support member branches are integrally formed with and extend proximally from the support member hub 224. For example, the entire support member 200 may be cut from a single sheet of material using conventional manufacturing techniques. This unitary structure provides robust structural properties, for example, selective flexibility and enhanced fatigue characteristics, particularly in areas that are subject to relatively high stresses during manufacture and use of the electroporation catheter 200. Forming the support member 220 from a superelastic material such as a nickel-titanium alloy facilitates configuring the support member 220 to assume its desired unconstrained shape such as shown in FIG. 2A due to the shape memory properties of the material, while providing sufficient flexibility necessary to collapse the electrode assembly 210 within a delivery sheath. In embodiments, the support member branches can be selectively configured along their lengths to tune the mechanical characteristics of the electrode assembly 210.

The flexible circuit 222 includes a flex circuit hub 230 and a plurality of flex circuit branches 234A-234F. In embodiments, the flex circuit hub 230 is disposed over and secured to the support member hub 224. In embodiments, the flex circuit branches 234A-234F are integrally formed with the flex circuit hub 230, and each of the flex circuit branches 234A-234F is disposed over and secured to a respective one of the support member branches. The flexible circuit 222 comprises a layered construction including one or more dielectric substrate layers, and conductive traces formed thereon. Similar to the support member 220, the unitary construction of the flexible circuit 222 enhances its structural properties, for example, by minimizing joints or other discontinuities at regions subject to relatively high stresses during use.

As shown, the flexible circuit 222 includes an ablation electrode 238 that has an ablation electrode hub portion 240 and a plurality of ablation electrode branches 242A-242F. In the illustrated embodiment, the distal ablation electrode hub portion 240 is located on the flex circuit hub 230. Additionally, the ablation electrode branches 242A-242F are integrally formed with the ablation electrode hub portion 240. Each of the ablation electrode branches 242A-242F extends proximally along a portion of a respective one of the flex circuit branches 234A-234F.

As further shown, the flexible circuit 222 includes a plurality of spline sensing electrodes 250. In the illustrated embodiment, two of the spline sensing electrodes 250 are disposed within a periphery of each of the ablation electrode branches 242A-242F, and one of the spline sensing electrodes 250 is located proximal to each of the ablation electrode branches 242A-242F on a respective flex circuit branch 234A-234F. The illustrated configuration is exemplary only, and other embodiments of the catheter 200 may have alternative configurations. Thus, in various embodiments, one or more of the spline sensing electrodes 250 may be disposed within the periphery of one or more of the ablation electrode branches 242A-242F and electrically isolated therefrom, and one or more of the spline sensing electrodes 250 may be located proximal to the ablation electrode branches 242A-242F on the respective flex circuit branch 234A-234F. In still other embodiments, no spline sensing electrodes 250 may be located outside the peripheries of the ablation electrode branches 242A-242F.

In some embodiments, the structural functionality of the support member 220 can be provided by a suitably designed flexible circuit 222. As such, although the electrode assembly 210 is described in detail as including the support member 220 as a primary structural member, in other embodiments the support member 220 can be omitted in its entirety and the corresponding functionality can be provided by the flexible circuit 222.

In the particular illustrated embodiment, the electroporation catheter 200 includes a pair of shaft electrodes 256 located proximate the distal end 209 of the outer shaft 202, as well as a central post 258 extending distally from the distal end 209 of the outer shaft 202. As shown, the central post 258 extends partially into the inner space 212, and includes a post electrode 260. In embodiments, the central post 258 may house additional components. For example, in embodiments, a magnetic navigation sensor (not shown) may be partially or wholly disposed within the central post 258. However, in other embodiments such a sensor may be located elsewhere on the electroporation catheter 200 (e.g., within the outer shaft 202). In the illustrated embodiment, the electrode assembly 210 further includes a hub sensing electrode 264 centrally located on the flex circuit hub 230.

In embodiments, one or both of the shaft electrodes 256 can be configured to be paired with the ablation electrode 238 to form an anode/cathode ablation electrode pair for generation of an ablative electric field in a bipolar mode. In embodiments, the shaft electrodes 256 may have additional functions, e.g., and without limitation, as additional sensing electrodes for sensing cardiac electrical signals, and for use as localization sensors for impedance tracking of the electrode assembly 210.

The post electrode 260 can provide a number of functional advantages. In one example, the post electrode 260 can operate as a reference for unipolar electrograms, in lieu of reliance on surface ECG patch electrodes as are otherwise known in the art. The location of the post electrode 260 for this purpose positions the reference electrode much closer to the tissue being sensed than is possible with the conventional surface ECG approach, which may advantageously minimize far field noise and provide much sharper unipolar electrograms than what are possible using surface ECG electrodes. The post electrode 260 may also be operable to sense and measure other electrical parameters, e.g., voltages between it and the ablation electrodes or other sensing electrodes on the electrode assembly 210, thereby providing data usable for, in some examples, determining the shape of the electrode assembly during use (including when deformed by forces applied by cardiac walls), and displaying shape information via the EAM system 70 (FIG. 1).

In embodiments, the hub sensing electrode 264 allows tissue surface mapping to be conducted in a “forward” manner, eliminating the need to manipulate the electrode assembly 210 to place the spline sensing electrodes 250 against or proximate the tissue to be mapped. The inclusion of the hub sensing electrodes 264 further enhances bipolar sensing capabilities by providing for, in the illustrated embodiment, six additional bi-poles when paired with any of the distal-most spline sensing electrodes 250.

FIG. 2D is an enlarged plan view of a portion of the spline 216A, the ablation electrode branch 242A, and the flex circuit branch 234A, according to embodiments of the present disclosure. The structural features illustrated in FIG. 2D are representative the splines 216A-216F, the ablation electrode branches 242A-242F and the flex circuit branches 234A-234F.

As shown, the distal end portion 217A of the spline 216A has a maximum width WD, and the intermediate portion 219A of the spline 216A has a maximum width WI that is greater than the maximum width WD of the distal end portion. In the particular embodiment shown, the intermediate portion 219A further includes one or more scalloped regions 272 wherein the opposing outer edges of the spline 216A have a concave shape. In embodiments, the scalloped regions 272 are selectively located along the length of the spline 216A and each have a scalloped region minimum width WS that is less than the maximum width WI of the intermediate portion 219A. When present, the scalloped regions 272 affect the mechanical properties (e.g., bending flexibility) of the spline 216A, to, for example, facilitate deformation of the spline 216A when it is in contact with target tissue, as well as facilitating collapse of the electrode assembly 210 when it is retracted into a delivery sheath. However, in some embodiments, the scalloped regions 272 are omitted, and the spline 216A has a generally linear shape along the intermediate portion 219A. In the illustrated embodiment, at least one of the scalloped regions 272 is located in the region of the spline 216A on which a portion of the ablation electrode branch 242A is disposed, and between the spline sensing electrodes 250 located thereon.

As shown, the ablation electrode branch 242A has a proximal end 274A. In the illustrated embodiment, the proximal end 274A is contoured and shaped to enhance electric field generation and clinical efficacy when the catheter 200 is configured to operate in bi-polar energy delivery mode, with the ablation electrode 238 and one or both of the shaft electrodes 256 paired as a bi-polar electrode pair. In other embodiments, however, the proximal end 274A can take on different shapes, e.g., semi-circular. The location of the proximal end 274A (which as will be appreciated, defines the length of the ablation electrode branch 242A and consequently defines, in part, the overall surface area of the ablation electrode 238) can be varied from embodiment to embodiment depending on the particular clinical needs required of the catheter 200.

As further shown, the ablation electrode branch 242A includes a plurality of ablation electrode branch apertures 278, and one of the spline sensing electrodes 250 is disposed within each of the ablation electrode branch apertures 278.

Application of high voltage pulsed field ablation energy to the ablation electrode 238 creates a high strength electrical field. The support member 220 is disposed in the high strength electrical field. In cases in which a conductive material is used as a stiffener in the support member 220, undesirable electrical coupling (via capacitance or some other mechanism) between the conductive support member 220 and the flex circuit may occur. Such electrical coupling can result in localized heating of the ablation electrode 238 and the sense electrodes 250. Accordingly, electrical coupling between the flex circuit and the conductive support member 220 is to be avoided to maintain a viability of the flexible-circuit-based electroporation catheter architecture.

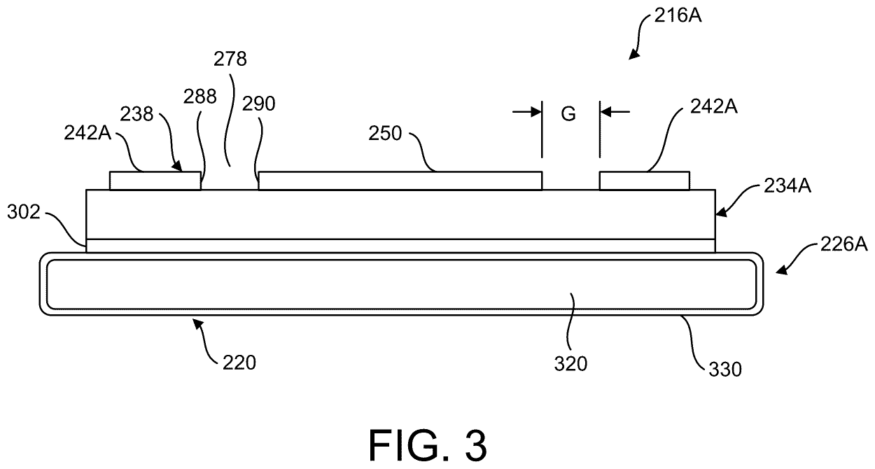

FIG. 3 is a schematic cross-sectional view of the spline 216A taken along the line 3-3 in FIG. 2D, illustrating an exemplary configuration of the flex circuit branch 234A disposed on the support member branch 226A on the spline 216A. In embodiments, the particular design of the flex circuit branch 234A (and the flex circuit as a whole) can be tailored for the particular clinical needs present. In the particular embodiment illustrated in FIG. 3, the flex circuit branch 234A is secured to the support member branch 226A by an adhesive layer 302, which may be any suitable adhesive.

As further shown in FIG. 3, the ablation electrode 238 and the spline sensing electrode 250 are disposed on an upper surface of the flex circuit branch 234A. In embodiments, both the ablation electrode and the sensing may have a coating of a suitable biocompatible metal, e.g., gold. In embodiments, the outer surfaces of the electrodes may be treated to provide the electrical properties desired for the particular clinical application.

As illustrated in FIG. 3, the proximal ablation electrode aperture 278 is bounded by an inner peripheral surface 288 of the ablation electrode branch 242A, and an outer peripheral surface 290 of the spline sensing electrode 250 is spaced from the inner peripheral surface 288 of the ablation electrode branch 242A by a gap G. In some embodiments, the gap G and portions of the ablation electrode 238 and the spline sensing electrode 250 may be selectively covered by a dielectric material (not shown).

FIG. 3 further illustrates one embodiment of a support member 220 configured to reduce the likelihood of electrically coupling between the support member 220 and the flex circuit branch 234A. The support member 220, as illustrated via support member branch 226A, includes an electrically conductive base member 320 covered with an electrically insulative coating 330. In the illustrated example, the electrically insulative coating 330 is a thin film of a dielectric material such as silicone, parylene, polyvinylidene fluoride, or other materials having similar dielectric properties. In one embodiment, the electrically insulative coating 330 is deposited on the base member 320 via an appropriate process including spay coat, dip coat, chemical vapor deposition, and atomic layer deposition, and the like. In one embodiment, the electrically insulative coating 330 encapsulates the entire electrically conductive base member 320 distal to the shaft distal end 209 (see FIG. 2A).

The thicknesses of the conductive base member 320 and the coating 330 may be selectively tailored to provide a desired degree of structural support and the aforementioned electrical decoupling. In one exemplary embodiment, the base member 320 may have a thickness of about 68 micrometers, and the dielectric coating 330 may have a thickness of about 12 micrometers, such that the overall thickness of the support member branch 226A is about 92 micrometers.

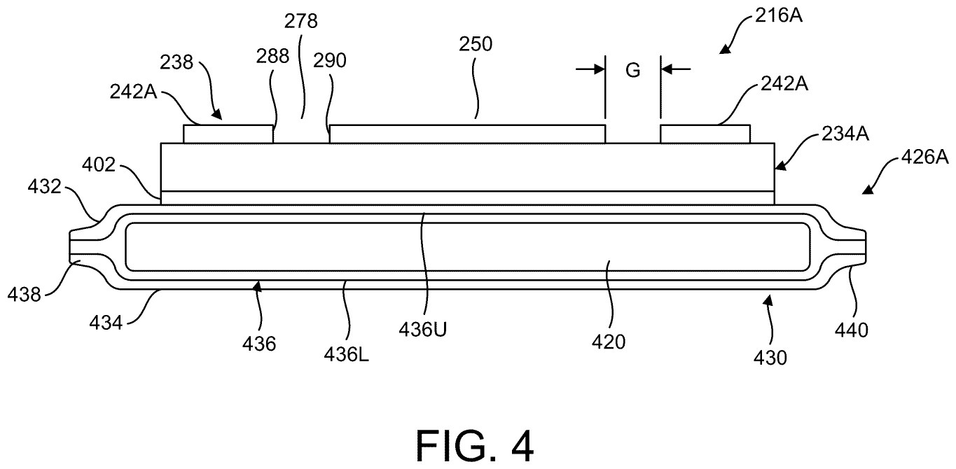

FIG. 4 is a schematic cross-sectional view similar to FIG. 3 of the spline 216A taken along the line 3-3 in FIG. 2D, but FIG. 4 illustrates an exemplary configuration of the flex circuit branch 234A disposed on another configuration of support member branch 426A. The flex circuit branch 234A comprises a layered structure that, except as specifically distinguished herein, may be typical of flexible circuits for use in medical device electrode assemblies. For example, the flex circuit branch 234A is affixed to the support member branch 426 via an adhesive layer 402. As further shown in FIG. 4, the ablation electrode branch 242A and the spline sensing electrode 250 are disposed on an upper surface of flex circuit branch 234A. As illustrated in FIG. 4, the proximal ablation electrode aperture 278 is bounded by an inner peripheral surface 288 of the ablation electrode branch 242A, and an outer peripheral surface 290 of the spline sensing electrode 250 is spaced from the inner peripheral surface 288 of the ablation electrode branch 242A by a gap G. In some embodiments, the gap G and portions of the ablation electrode 238 and the spline sensing electrode 250 may be selectively covered by a dielectric material (not shown).

FIG. 4 further illustrates another embodiment of support member 220 configured to reduce the likelihood of electrically coupling with a high strength electrical field of the ablation electrode 238. The illustrated embodiment of support member branch 426A includes an electrically conductive base member 420 covered with an electrically insulative coating 430. In the illustrated example, the electrically insulative coating 430 has a laminated construction and encapsulates and electrically insulates the electrically conductive base member 420. In the illustrated example, the electrically insulative coating 430 includes an upper dielectric layer 432, a lower dielectric layer 434, and an adhesive layer 436 including a lower adhesive layer portion 436L and an upper adhesive layer portion 436U. As further shown, the upper and lower adhesive layer portions 436U, 436L are directly disposed on the base member 420, and the dielectric layers 432, 434 are disposed over the upper adhesive layer portion 436U and the lower adhesive layer portion 436L, respectively. Additionally, the dielectric coating 430 includes lateral extensions 438, 440 extending laterally relative to the base member 420, whereby the adhesive layer 436 is effectively sandwiched between the upper and lower dielectric layers 432, 434 in the lateral extensions 438, 440. In embodiments, the upper and lower adhesive layer portions 436U, 436L function to secure the insulative coating 430 to the base member 420 as well as to prevent delamination of the dielectric layers 432, 434, and the extensions 438, 440 result from the lamination of the dielectric layers 432, 434 to the material forming the adhesive layer 430.

In embodiments, each of the dielectric layers 432, 434 is comprised of a film of dielectric material, such as polyimide or liquid crystal polymer. In embodiments, the adhesive layer 436 may be composed of an acrylic adhesive film, an epoxy adhesive, or other similar adhesives.

Similar to the embodiment of FIG. 3, the thicknesses of the conductive base member 420 and the coating 430 may be selectively tailored to provide a desired degree of structural support and the aforementioned electrical decoupling. In one exemplary embodiment, the conductive base member 420 has a thickness of about 68 micrometers, and the dielectric layers 432, 434 and the upper and lower adhesive layer portions 436U, 436L each have a thickness of about 12 micrometers, resulting in an overall thickness of about 116 micrometers.

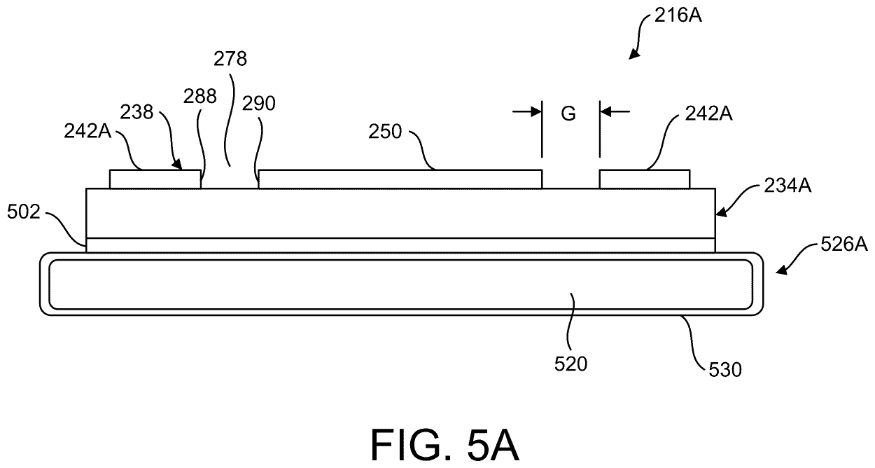

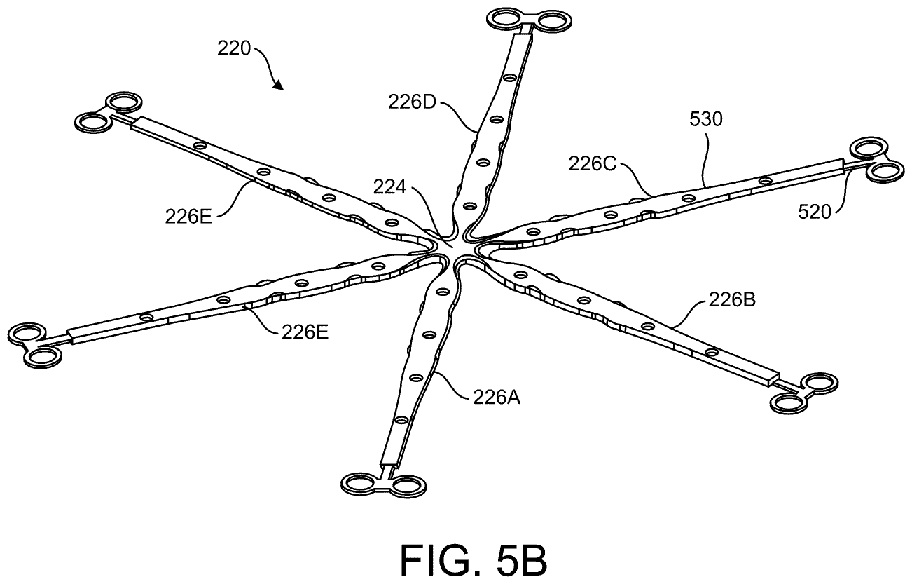

FIG. 5A is a schematic cross-sectional view similar to FIG. 3 of the spline 216A taken along the line 3-3 in FIG. 2D, but FIG. 5A illustrates an exemplary configuration of the flex circuit branch 234A disposed on still another configuration of support member branch 526A. The flex circuit branch 234A comprises a layered structure that, except as specifically distinguished herein, may be typical of flexible circuits for use in medical device electrode assemblies. In embodiments, the flex circuit branch 234A is affixed to the support member branch 526 via an adhesive layer 502. As further shown in FIG. 5, the ablation electrode branch 242A and the spline sensing electrode 250 are disposed on an upper surface of flex circuit branch 234A. As illustrated in FIG. 5, the proximal ablation electrode aperture 278 is bounded by an inner peripheral surface 288 of the ablation electrode branch 242A, and an outer peripheral surface 290 of the spline sensing electrode 250 is spaced from the inner peripheral surface 288 of the ablation electrode branch 242A by a gap G. In some embodiments, the gap G and portions of the ablation electrode 238 and the spline sensing electrode 250 may be selectively covered by a dielectric material (not shown).

FIG. 5A further illustrates still another embodiment of support member 220 configured to reduce the likelihood of electrically coupling with a high strength electrical field of the ablation electrode 238. The illustrated embodiment of support member branch 526A includes an electrically conductive base member 520 covered with an electrically insulative coating 530. In the illustrated example, the electrically insulative coating 530 is a dielectric material overmolded on the electrically conductive base member 520 to encapsulate and insulate the electrically conductive base member 520. In embodiments, the overmolded electrically insulative coating 530 is formed of a moldable elastomeric material. Examples of the electrically insulative coating 530 may include, without limitation, silicone, urethane, and a polyether block amide that, in some examples, is available under the trade designations PEBAX from Arkema, S. A., and VESTAMID E from Evonik Industries, AG. In one example, the flex circuit branch 234A is affixed to the electrically insulative overmolded coating 530 via the adhesive layer 502.

FIG. 5B is an isometric illustration of the support member 220 having an electrically conductive base member 520 covered with an electrically insulative overmolded coating 530 illustrated in FIG. 5A. The support member 220 includes a support member hub 224 and a plurality of support member branches 226A-226F. In embodiments, the support member branches 226A-226F are integrally formed with and extend proximally from the support member hub 224. For example, the entire support member 220 may be constructed from a single sheet of nitinol base member 520 using conventional manufacturing techniques and an electrically insulative cover 530 is applied to the base member. This unitary nitinol structure provides robust structural properties, for example, selective flexibility and enhanced fatigue characteristics, particularly in areas that are subject to relatively high stresses during manufacture and use of the electroporation catheter 200. Forming the support member 220 from a superelastic base material such as a nitinol covered in a polyether block amide facilitates configuring the support member 220 to assume its desired unconstrained shape such as shown in FIG. 2A due to the shape memory properties of the base material, while providing sufficient flexibility necessary to collapse the electrode assembly 210 within a delivery sheath as well as electrically insulating the nitinol from a high strength electric field. In embodiments, the support member branches 226A-226F can be selectively configured along their lengths to tune the mechanical characteristics of the electrode assembly 210. As shown, in embodiments, the overmolded cover 530 may include various features, e.g., apertures and tabs, to facilitate positioning and attachment of the flex circuit to the support member 220.

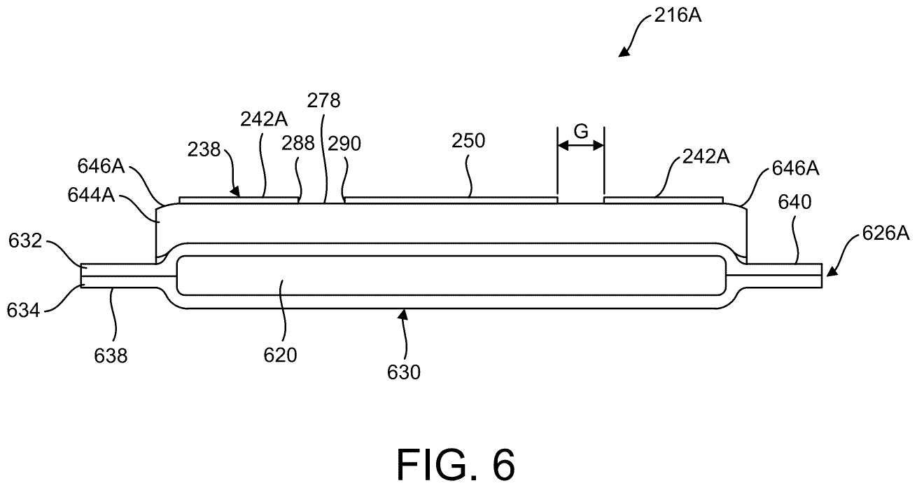

FIG. 6 is a schematic cross-sectional view similar to FIG. 3 of the spline 216A taken along the line 3-3 in FIG. 2D, but FIG. 6 illustrates an exemplary configuration of another configuration of flex circuit branch 644A disposed on another configuration of support member branch 626A. The flex circuit branch 644A comprises a liquid crystal polymer (LCP) material that can be reflowed, unlike a polyimide material that cannot be reflowed. Thus, during manufacturing of the spline 216A, the LCP material of the flex circuit branch 644A is reflowed to secure the flex circuit branch 644A to the support member branch 626A. The result is the elimination of a separate adhesive layer therebetween, so the flex circuit branch 644A is mechanically bonded to the support member branch 626A instead of being chemically bonded. Such a configuration has the benefits of preventing delamination, offering better support for the electrode traces, reducing the component count of the assembly, reducing thrombogenicity of the spline 216A, and allowing micro-scale translation between the flex circuit branch 644A and the support member branch 626A, which improves the shape retention properties of the spline 216A. Furthermore, reflowing the flex circuit branch 644A rounds the edges 646A of the flex circuit branch 644A so there is a softer tissue interaction from the spline 216A.

As further shown in FIG. 6, the ablation electrode branch 242A and the spline sensing electrode 250 are disposed on an upper surface of flex circuit branch 644A. As illustrated in FIG. 6, the proximal ablation electrode aperture 278 is bounded by an inner peripheral surface 288 of the ablation electrode branch 242A, and an outer peripheral surface 290 of the spline sensing electrode 250 is spaced from the inner peripheral surface 288 of the ablation electrode branch 242A by a gap G. In some embodiments, the gap G and portions of the ablation electrode 238 and the spline sensing electrode 250 may be selectively covered by a dielectric material (not shown).

FIG. 6 further illustrates still another embodiment of an electrically conductive base member 620 configured to reduce the likelihood of electrically coupling with a high strength electrical field of the ablation electrode 238. The illustrated embodiment of support member branch 626A includes the base member 620 covered with an electrically insulative coating 630. In the illustrated example, the electrically insulative coating 630 has a mechanically bonded construction and encapsulates and electrically insulates the base member 620. In the illustrated example, the electrically insulative coating 630 includes an upper LCP layer 632 and a lower LCP layer 634. Thereby, the upper and lower LCP layers 632, 634 are in direct contact with the base member 620 and form the exterior of the electrically insulative coating, and the upper LCP layer 632 is in direct contact with and bonded directly to the flex circuit branch 644A. Additionally, the electrically insulative coating 630 includes lateral extensions 638, 640 extending laterally relative to the base member 620. In embodiments, the upper and lower LCP layers 632, 634 are reflowed to secure the electrically insulative coating 630 to the base member 620 as well as to prevent delamination of the upper and lower LCP layers 632, 634. In embodiments, the extensions 638, 640 result from the bonding of the upper and lower LCP layers 632, 634 around the base member 620.

Similar to the embodiment of FIG. 3, the thicknesses of the conductive base member 620 and the electrically insulative coating 630 may be selectively tailored to provide a desired degree of structural support and the aforementioned electrical decoupling. In one exemplary embodiment, the base member 620 comprises a nitinol material and has a thickness of about 84 micrometers, and the upper and lower LCP layers 632, 634 each have a thickness of about 25 micrometers. In such an embodiment, the overall thickness of the support member branch 626A is about 134 micrometers.

In the various embodiments of the disclosure, the electrically conductive support members may be electrically connected in parallel with as the corresponding ablation electrode of the electrode assembly. For example, in the illustrated examples of the support member branches 226A, 426A, 526A, 626A depicting configurations of the support member 220, the electrically conductive base members 320, 420, 520, 620 are electrically coupled in parallel to the flexible circuit ablation electrode 238, such as within the shaft 208 or on the electrode assembly itself. Electrically coupling the conductive base member to the ablation electrode functions to prevent capacitive coupling mechanism of the electrically conductive support member and the flexible circuit. Because the conductive base member and the electrode are at the same potential, but because the base member is sufficiently electrically insulted at therapy voltage levels, current only flows out of the flexible circuit electrode, which removes or reduces the burden of the electrical insultation of the dielectric coating.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112 (f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A catheter for use in ablating cardiac tissue through irreversible electroporation, the catheter comprising:
a tubular outer shaft having a proximal end and an opposite distal end; and
an electrode assembly extending distally from the distal end of the outer shaft, the electrode assembly defining a distally located central hub portion and a plurality of splines each including a distal end portion extending proximally from the central hub portion, and a proximal end portion attached to and constrained by the outer shaft, the electrode assembly comprising:
a flexible circuit having a flex circuit hub and a plurality of flex circuit branches integrally formed with and extending proximally from the flex circuit hub, the flexible circuit further including an outwardly-facing ablation electrode including a plurality of ablation electrode branches extending proximally along a portion of a respective one of the flex circuit branches and terminating in an ablation electrode proximal end; and
a support member having a support member hub and a plurality of support member branches extending proximally from the support member hub, wherein the support member includes an electrically conductive base member covered by an electrically insulative coating, wherein each of the flex circuit branches is secured to a respective one of the support member branches, and the ablation electrode is electrically coupled to the electrically conductive base member.

2. The catheter of claim 1, wherein the electrically insulative coating comprises a silicone, parylene, or polyvinylidene fluoride coating, and the electrically insulative coating is deposited via a process including spray coat, dip coat, chemical vapor deposition, and atomic layer deposition.

3. The catheter of claim 1, wherein the electrically insulative coating has a laminated structure including an upper dielectric layer, a lower dielectric layer, and an adhesive layer.

4. The catheter of claim 3, wherein the adhesive layer includes an upper adhesive layer portion disposed over an upper surface of the conductive base member, and a lower adhesive layer portion disposed over a lower surface of the conductive base member, wherein the upper dielectric layer is disposed over the upper adhesive layer portion, and the lower dielectric layer is disposed over the lower adhesive layer portion.

5. The catheter of claim 4, wherein the upper and lower dielectric layers each comprise a polyimide film, the upper and lower adhesive layer portions each comprise an acrylic adhesive film, and the upper and lower dielectric layers and the adhesive layer form dielectric coating extensions extending laterally from opposite lateral edges of the conductive base member.

6. The catheter of claim 1, wherein the electrically insulative coating is a polyether block amide or silicone that is overmolded to the electrically conductive base member.

7. The catheter of claim 1, wherein the plurality of flex circuit branches comprises a liquid crystal polymer material.

8. The catheter of claim 1, wherein the electrically insulative coating comprises a liquid crystal polymer material and the electrically insulative coating is an upper layer that is mechanically bonded to a lower layer using a process of reflowing the liquid crystal polymer material.

9. The catheter of claim 8, wherein the upper and lower dielectric layer are in direct contact with the conductive base member, and the upper dielectric layer is in direct contact with the plurality of flex circuit branches.

10. A catheter for use in ablating cardiac tissue through irreversible electroporation, the catheter comprising:
a tubular shaft; and
a splined electrode assembly comprising:
a support member comprising an electrically conductive base member covered by an electrically insulative coating; and
a flexible circuit secured to the support member, the flexible circuit having a dielectric substrate layer and an ablation electrode disposed on the dielectric substrate layer, wherein the ablation electrode is electrically coupled to the conductive base member.

11. The catheter of claim 10, wherein the electrically insulative coating includes an upper adhesive layer portion disposed over an upper surface of the conductive base member, a lower adhesive layer portion disposed over a lower surface of the conductive base member, an upper dielectric layer disposed over the upper adhesive layer portion, and a lower dielectric layer disposed over the lower adhesive layer portion.

12. The catheter of claim 11, wherein the upper and lower dielectric layers each comprise a polyimide film or a liquid crystal polymer film, and the upper and lower adhesive layer portions each comprise an acrylic adhesive film.

13. The catheter of claim 10, wherein the electrically insulative coating is overmolded to the electrically conductive base member, and the electrically insulative coating comprises a polyether block amide or silicone coating.

14. The catheter of claim 10, wherein the plurality of flex circuit branches comprises a liquid crystal polymer material.

15. The catheter of claim 10, wherein the electrically insulative coating comprises a liquid crystal polymer material and the electrically insulative coating is an upper layer that is mechanically bonded to a lower layer using a process of reflowing the liquid crystal polymer material.

16. The catheter of claim 15, wherein the upper and lower dielectric layer are in direct contact with the conductive base member, and the upper dielectric layer is in direct contact with the plurality of flex circuit branches.

17. A catheter for use in ablating cardiac tissue through irreversible electroporation, the catheter comprising:
a tubular shaft; and
an electrode assembly extending from the tubular shaft, the electrode assembly comprising a plurality of splines extending proximally from a distal hub portion, the distal hub portion and the plurality of splines comprising:
a support member comprising an electrically conductive base member, and an electrically insulative coating disposed over the electrically conductive base member; and
a flexible circuit comprising a dielectric substrate layer secured to the electrically insulative coating, and an ablation electrode disposed on the dielectric substrate layer, wherein the ablation electrode is electrically coupled to the conductive base member.

18. The catheter of claim 17, wherein the electrically insulative coating has a laminated structure including an upper adhesive layer disposed on an upper surface of the conductive base member, a lower adhesive layer disposed on a lower surface of the conductive base member, an upper dielectric layer disposed on the upper adhesive layer, and a lower dielectric layer disposed on the lower adhesive layer.

19. The catheter of claim 18, further comprising dielectric coating extensions extending laterally from opposite lateral edges of the conductive base member, each of the dielectric coating extensions comprising an extension of the upper and lower dielectric layers with the upper and lower adhesive layers disposed therebetween, wherein the upper and lower dielectric layers each comprise a polyimide film or a liquid crystal polymer film, and the upper and lower adhesive layers each comprise an acrylic adhesive film.

20. The catheter of claim 17, wherein the electrically insulative coating comprises a liquid crystal polymer material, and the electrically insulative coating is an upper layer that is mechanically bonded to a lower layer using a process of reflowing the liquid crystal polymer material, and the upper and lower dielectric layer are in direct contact with the conductive base member.
